(19) United States
(12) Patent Application Publication
(10) Pub. No.: US 2013/0224119 A1
(43) Pub. Date: Aug. 29, 2013

(54) OMENTUM AND USE THEREOF

(71) Applicant: Surinder Singh SAINI, Newport Beach, CA (US)

(72) Inventor: Surinder Singh SAINI, Newport Beach, CA (US)

(21) Appl. No.: 13/849,014

(22) Filed: Mar. 22, 2013

Related U.S. Application Data
(63) Continuation of application No. 13/309,468, filed on Dec. 1, 2011, now abandoned.
Application No. 13/309,468 is a continuation of application No. 12/361,808, filed on Jan. 29, 2009, now abandoned.
Application No. 12/361,808 is a continuation of application No. PCT/US09/31462, filed on Jan. 20, 2009.
(60) Provisional application No. 61/023,840, filed on Jan. 26, 2008.
(60) Provisional application No. 61/113,937, filed on Nov. 12, 2008.

(51) Int. Cl. A61K 35/38; A61B 5/00

(52) U.S. Cl. CPC A61K 35/38; A61B 5/4848
USPC 424/9.2; 424/93.7; 514/626; 435/325; 600/300

(57) ABSTRACT

In some embodiments, mammalian omentum and methods of using the same for treating symptoms and/or conditions related to dementia are provided. In addition, a composition comprising the omentum tissue and/or the extract thereof is described in some embodiments. In some other embodiments, methods of formulating and administering the composition are also provided. In addition, a cultured cell system to culture cells and/or tissues of the omentum is described. Some aspects of embodiments provide methods of identifying one or more biological agent from the omentum tissue. In another aspect of the embodiments, methods of testing the effect of stimulation omentum in treating dementia conditions are described.

### BACKGROUND OF THE INVENTION

1. Field of the Invention

The present disclosure generally relates to omentum and methods of using omentum tissue and/or an extract of the omentum tissue to treat symptoms and/or conditions associated with dementia. In some aspects of embodiments, methods of identifying one or more biological agent from the omentum tissue or an extract thereof are provided.

2. Description of the Related Art

Dementia generally refers to a group of symptoms and/or conditions associated with the decline of cognition function. The areas of cognition affected by dementia include, but are not limited to, memory, attention, language, problem solving and emotion. Dementia is generally considered to occur at least in part due to brain dysfunction, and extensive research has been performed to uncover the disease mechanisms and develop means to cure the disease.

Omentum generally refers to a part of peritoneum encompassing a variety of internal structures. For last 30 years or more, the surgical transposition of omentum to the brain has been shown to induce noticeable improvement in dementia conditions from several cases. However, such surgical approaches are substantially invasive and therefore often cause unnecessary complications to patients.

### SUMMARY OF THE INVENTION

Disclosed herein are methods of identifying a biological agent for treating dementia which may comprise obtaining an aqueous or organic extract of omentum tissue, separating the extract into two or more fractions, testing an activity of each of the fractions for an indication of its ability to treat dementia, selecting at least one fraction that has the indication of ability to treat dementia, and identifying one or more biological agents for treating dementia from said at least one fraction that has the indication of ability to treat dementia. In the foregoing method, said biological agent can be a neurotrophic agent. In the foregoing method, said testing an activity of each of the fractions for an indication of its ability to treat dementia may be done in vivo or in vitro.

One aspect of the disclosure provides a method of growing a mammalian omentum cell and/or tissue outside a mammal body which may comprise providing an omentum cell and/or tissue obtained from a mammal, and growing the mammalian omentum cell and/or tissue in a cell culture system.

Another aspect of the disclosure provides a composition comprising an omentum tissue or an aqueous or organic extract of an omentum tissue, and at least one pharmaceutically acceptable carrier selected from the group consisting of water, oil, albumin, a filler, a disintegrant, a lubricant, a glidant, and a binder. In the foregoing composition, the omentum tissue or extract of an omentum tissue can be obtained from a mammal or from a mammalian omentum cell culture system. In the foregoing composition, a target disease of said composition may comprise dementia. The foregoing composition can be administered to a mammal via a means of oral administration, parenteral administration, inhalation, topical administration, rectal administration, vaginal administration, implantation, or any combinations thereof.

Still another aspect of the disclosure provides a method of formulating a composition which may comprise providing an omentum tissue or an aqueous or organic extract of an omentum tissue, mixing the omentum tissue or the extract of an omentum tissue with at least one pharmaceutically acceptable carrier selected from the group consisting of water, oil, albumin, a filler, a disintegrant, a lubricant, a glidant, and a binder, and making a mixture of said omentum tissue or extract of an omentum tissue with at least one pharmaceutically acceptable carrier into a form suitable for oral administration, parenteral administration, inhalation, topical administration, rectal administration, vaginal administration, implantation, or any combinations thereof.

Further, some aspects of the disclosure provide a method of treating dementia comprising administering a composition, wherein said composition may comprise an omentum tissue or an aqueous or organic extract of an omentum tissue to a mammal. In the foregoing method of treating dementia, the composition may be administered to a mammal via a means of oral administration, parenteral administration, inhalation, topical administration, rectal administration, vaginal administration, implantation, or any combinations thereof.

Other aspects of the disclosure provide a method of treating dementia comprising providing a stimulant in or near omentum to a mammal In one example, the stimulant can be 1% lidocaine.

Some other aspects of the disclosure provide a method of treating dementia comprising implanting an omentum stimulator to a mammal. In some examples, the omentum stimulator can be implanted in or near omentum. In some other examples, the stimulator may be configured to deliver an omentum tissue and/or an aqueous or organic extract of an omentum tissue to a subcutaneous tissue, a subdural spinal tissue, a subdural cranial tissue or any combinations thereof.

Still some other aspects of the disclosure provide a method of testing an effect of stimulating omentum on treating dementia which may comprise stimulating omentum in a first subject, optionally stimulating tissue other than omentum in a second subject, and measuring one or more symptom and/or condition related to dementia from the first and optional second subjects. In the foregoing method of testing, said one or more symptom and/or condition related to dementia may be measured via a behavioral approach, a neurological approach, a neuropsychological approach, a pathological approach, or any combinations thereof. In one example of the foregoing method of testing, stimulating omentum in the first subject may be applying a gastrostomy tube to or near omentum. In another example of the foregoing method of testing, said stimulator in the second subject may be an endoscopic tube and said endoscopic tube may be provided through a mouth.

### DETAILED DESCRIPTION OF THE PREFERRED EMBODIMENT

The present disclosure generally relates to omentum and methods of using omentum tissue and/or an extract of the omentum tissue to treat symptoms and/or conditions associated with dementia. In one embodiment, methods of identifying a biological agent from the omentum tissue or an extract thereof are provided.

Omentum generally refers to a part of the peritoneum encompassing a variety of internal structures. Examples of internal structures that are encompassed by omentum are liver, stomach, small and large intestines. Omentum usually comprises a thin layer comprising cellular and non-cellular tissues. Such cellular and non-cellular tissues include, but are not limited to, adipose tissue, connective tissue and any fluid filling between the folded layers and/or between omentum and internal structures. Omentum often includes nerves and/or vessels of blood and lymph as omentum serves as a conduit for neuronal and vascular tissues.

At least some part of the present disclosure is based on multiple observations by the inventor during his practices. As a gastrointestinal specialist, the inventor often needs to apply a gastrostomy tube to an abdominal area of a patient via some surgical procedures. In some cases when the patient having symptoms related to dementia underwent the surgery, the inventor observed that some symptoms became noticeably improved for at least 24 hours or longer after the surgery.

This observation has led the inventor to a hypothesis that stimulation of omentum can improve symptoms and/or conditions related to dementia. Moreover, such improvement may occur at least in part because the stimulation of omentum can induce production and/or secretion of one or more biological agent that can improve conditions associated with dementia.

In one embodiment, a method of testing an effect of stimulating omentum on treating dementia symptoms and/or conditions is provided. In one illustrative example, the method may comprise: stimulating omentum in a first subject, stimulating tissue other than omentum in a second subject, and measuring one or more symptom and/or condition related to dementia between the first and second subjects.

In another illustrative example, the method may comprise: stimulating omentum in a first subject, and measuring and comparing one or more symptom and/or condition related to dementia before and after the stimulation.

In these particular examples, the subject can be any kind of mammals including a human, a chimpanzee, a dog, a pig, a mouse, a rat, and a rabbit.

Stimulating omentum can be done in a variety of approaches. For example, a stimulant such as 1% lidocaine can be injected to or near omentum to stimulate omentum. Alternatively, a stimulator which is configured to mechanically, electrically and/or chemically stimulate omentum can be implanted to or near omentum. Mechanical stimulation can be, for example, vibration of omentum, suction of omentum, incision of omentum, and any combination thereof. Electrical stimulation can be, for example, providing electrical shock with about 0.1 voltage (V) to about 100 V for about several milliseconds to about several minutes to or near omentum. Chemical stimulation can be, for example, providing a stimulant such as 1% lidocaine to or near omentum.

In one illustrative example, a gastrostomy tube can be applied to or near omentum of a first subject. In a second subject if desired, an endoscopic tube can be applied through a mouth without penetrating an abdominal area.

In some aspects of embodiments, symptoms and/or conditions related to dementia can be measured at least in part via a behavioral approach, a neurological approach, a neuropsychological approach, a pathological approach, or any combination thereof. Some examples of such measurement methodology are described in references including Goldsmith and others (2003) and Shankle and others (2008). Some illustrative examples of the measurement via a behavioral approach and/or a neurological approach are to measure the easiness and pattern of daily life activities such as bathing, continence, dressing, feeding, toileting, ambulating, telephoning, housework, using money and others. Some non-limiting examples of the measurement via a neuropsychological approach include, but are not limited to, monitoring depth perception, color spreading, face processing, temper control, paired associated learning and others. Also some illustrative examples of the measurement via a pathological approach includes, but are not limited to, monitoring CT brain, autopsy of a brain, SPECT of a brain and others.

While several foregoing examples are presented to provide an illustrative description of some embodiments, it should be apparent to a person having ordinary skill in the art that a variety of modification and/or substitution can be made in the foregoing examples without abandoning the scope of the present disclosure. Therefore, any modification or substitution in the foregoing examples and any other methods available in the art that are aimed to test the effect of stimulation of omentum in treating dementia should not be excluded from the scope of the disclosure.

The biological agent induced by stimulation of omentum may include an agent that can act in cells in a brain to prevent or delay development of dementia. Alternatively, the biological agent may act on some tissues to induce production and/or secretion of other biological agents to improve dementia. Examples of such some tissues include, but are not limited to, an omentum, a brain, a pituitary gland, a hypothalamus, a thyroid gland, a parathyroid gland, an adrenal gland, a pineal gland, a thymus, a pancreas, and a gonad.

In some embodiments, the biological agent induced by stimulation of omentum may include one or more neurotrophin. A neurotrophin is generally a member of a family of proteins or modified proteins that can induce survival, development, growth and/or function of neuronal cells. With stimulation, omentum can induce production and/or secretion of one or more neurotrophin. Alternatively, stimulation of omentum can induce production and/or secretion of a biological agent which can in turn induce production and/or secretion of a neurotrophin from omentum or other tissues such as a brain, a pituitary gland, a hypothalamus, a thyroid gland, a parathyroid gland, an adrenal gland, a pineal gland, a thymus, a pancreas, and a gonad.

One aspect of the present disclosure provides a method of identifying such one or more biological agents induced by stimulation of omentum. The biological agent can be any compound present in a mammal such as a nucleic acid, a protein, a carbohydrate, a lipid, an ion, a metal or any combinations thereof (e.g. a DNA, a RNA, a microRNA, a ribozyme, a glycoprotein, a protein with a lipid moiety, and a protein conjugated with a metal or an ion, and others).

There are a variety of methods available in the art of identifying and/or isolating a biological agent from tissue. Numerous biochemical and/or physical methods for identifying and/or isolating a biological agent may include, bur are not limited to, a chromatography, an electrophoresis, a centrifugation, an immunoprecipitation, and any combinations thereof. Also various analysis methods such as mass spectrometry, several types of gas and/or liquid chromatography, nuclei acid or protein sequencing analysis and others are well known in the art. As the intrinsic nature and benefit of each method are revealed in the art, it should be feasible for a person having ordinary skill in the art to use or modify any available method to identify a biological agent from omentum without abandoning the scope of the disclosure. Therefore, a choice of a certain method, modification and/or substitution in any method, and combination of any method that are aimed to identify a biological agent from omentum tissue should be considered to be included in the scope of the disclosure.

One particular illustrative example of how to identify such biological agents from omentum may comprise: obtaining an aqueous or organic extract of omentum tissue, separating the extract into two or more fractions, testing an activity of each of the fractions for an indication of its ability to treat dementia, selecting at least one fraction that has the indication of ability to treat dementia, and identifying one or more biological agents for treating dementia from said at least one fraction that has the indication of ability to treat dementia.

Generally in the present disclosure, omentum tissue refers to tissue comprising the omentum. As the omentum generally comprises a thin layer, when the omentum is obtained from a mammal, surrounding non-omentum tissue may be obtained simultaneously. Such non-omentum tissue can be any connective tissue, a cellular or non-cellular tissue, any fluid, and at least part of an internal structure that are present near the omentum. In some embodiments, the omentum tissue obtained and analyzed for identification of a biological agent can comprise the omentum and non-omentum. In certain embodiments, the tissue obtained and analyzed for identification of a biological agent can comprise about 5, 10, 15, 20, 25, 30, 35, 40, 45, 50, 60, 70, 80, 90, or 100% omentum with about 95, 90, 85, 80, 75, 70, 65, 60, 50, 40, 30, 20, 10, or 0% non-omentum, respectively.

A mammal in at least some embodiments can be any kind of mammals including a human, a chimpanzee, a dog, a pig, a mouse, a rat, and a rabbit.

In some embodiments, an extract generally refers to a part or whole substance extracted from a certain tissue. The extract can be extracted from the tissue comprising omentum, which is referred to the omentum tissue herein some embodiments.

As the omentum covers a large area of the abdomen, omentum tissue can be obtained from many different abdominal areas. For example, the omentum tissue can be obtained from the abdominal area near a small intestine. Alternatively, the omentum tissue can be obtained from the abdominal area near a stomach. In some embodiments, the omentum tissue can be obtained from more than one abdominal area.

The omentum tissue can be provided in various ways available in the art. In one example, a doctor or an operator who performs surgical procedures can open the abdominal wall and expose the omentum. Once the omentum is exposed, at least a part of the omentum with or without the non-omentum tissue can be isolated by incision. Alternatively, a doctor or an operator can insert a tool to a mammal, wherein the tool is configured to collect tissue comprising at least part of the omentum. For example, a tool, which can make incision of tissue and suck at least part of the tissue, can be inserted into the abdominal wall of a mammal via minimally invasive procedures. Such tool may collect the tissue comprising the omentum with or without the non-omentum tissue.

While two illustrative foregoing examples of how to obtain the omentum tissue from a mammal are described herein, it should be apparent to a person skilled in the art that there can be a variety of modifications and/or substitutions at least in part of described procedures without abandoning the scope of the disclosure. In addition, any other methodology known in the art that is aimed to obtain tissue including the omentum from a mammal also should be included in the scope of the present disclosure.

As an alternative example of how to obtain omentum tissue to make the extract, a cell culture technique can be utilized. Tissue comprising omentum can be obtained from a mammal as described previously. The obtained tissue can be cultured in a petri-dish with various cell culture techniques available in the art. For instance, the obtained tissue can be finely chopped with a tool such as a tissue disruptor or with a biochemical method such as a trypsin treatment to separate cells and/or tissues. Then, cells and/or tissues can be grown in commercially available cell culture medium such as DMEM that can be purchased from Thermo Fisher Scientific Inc. and others. Various mammalian cell culture techniques are available in the art (e.g. see Basic cell culture protocols by C. Helgason and C. Miller, Humana Press, 2004) and can be applied without limiting the scope of the disclosure.

In some aspects of embodiments, the omentum tissue can be obtained from a mammal or a cultured cell system and such obtained tissue can be processed to provide an extract. One exemplary way to obtain the extract is homogenizing the omentum tissue and extracting at least some substance from the homogenized tissue. Homogenization generally refers to a process that involves breaking apart cells, tissues and/or non-cellular structures such as cellular organs (e.g. nucleus, mitochondria and others). Homogenization can generally be done by using a tool such as a homogenizer, a blender, an ultrasonic disruptor, a mechanical disruptor, a mortar and pestle and others. In some cases, the tissue can be quickly frozen under liquid nitrogen and destructed by a mortar and pestle to be in a substantially homogeneous condition. In some other cases, the tissue obtained from a mammal can be homogenized without being treated with liquid nitrogen. In one example, homogenization can be done until the tissue becomes substantially homogenous.

At least in some aspects of embodiments, the homogenized tissue can be divided into a substantially aqueous extract and a substantially solid extract. Such division can be done via various ways available in the field including, for example, a centrifugation. In one case, the homogenized tissue can be centrifuged at about 1,000 rpm to about 15,000 rpm for about few seconds to about few hours. In some embodiments, homogenized tissues can be divided by being centrifuged at about 1,000 rpm, 2,000 rpm, 3,000 rpm, 4,000 rpm, 5,000 rpm, 6,000 rpm, 7,000 rpm, 8,000 rpm, 9,000 rpm, 10,000 rpm, 11,000 rpm, 12,000 rpm, 13,000 rpm, 14,000 rpm or 15,000 rpm for about 5 seconds, 10 seconds, 15 seconds, 30 seconds, 1 minute, 2 minutes, 5 minutes, 10 minutes, 30 minutes, 45 minutes, 1 hours, 1.5 hour, 2 hours, 3 hours, 4 hours or 5 hours. In one case, both substantially aqueous part and substantially solid part can be further separated into two or more fractions as described below. Alternatively, the substantially aqueous extract can be further processed to be separated into two or more fractions.

In an alternative case, obtaining an extract can be done at least via using a liquid-liquid extraction method, which is known in the art. For example, an aqueous solution such as water and an organic solution such as benzene can be mixed with the homogenized or non-homogenized omentum tissue. The omentum tissue can be obtained from a mammal or a cultured cell system. A certain compound present in the tissue can be displaced either in the water layer or the benzene layer according to its solubility. Once division of the relatively aqueous extract in the water layer and the relatively organic extract in the benzene layer is done, each extract can be separately collected. In one example, each extract can be further processed to be separated into two or more fractions. In another example, only one extract which is either a relatively aqueous extract or a relatively organic extract can be further processed to be separated into two or more fractions. While a liquid-liquid extraction method is described herein as an illustrative example of how to obtain an aqueous and/or organic extract, it should be apparent to a person skilled in the art that there are a variety of ways to modify and/or substitute the extraction procedures without abandoning the scope of the disclosure. Also there are other techniques or methods available in the art that are aimed to obtain an extract from tissue and such methods should be considered to be included in the scope of the disclosure.

In some embodiments, once the extract of the omentum tissue is obtained, the extract can be processed to be separated into two or more fractions. Such fractionation can be done via various ways available in the art. As an illustrative example, the fractionation of the extract can be done at least in part via a density gradient centrifugation method, a chromatography method, or a combination thereof. In some embodiments, a variety of chromatographic techniques that can separate fractions according to a mobile phase (Gas and/or liquid chromatography), affinity (affinity chromatography), size (size exclusion chromatography), ionic charge (ion exchange chromatography) and others can be applied along with other fractionation methods such as a centrifugation.

In certain embodiments, testing an activity of each of the fractions for an indication of its ability to treat dementia is provided. There are several assays of testing a potential activity of a fraction in treating dementia in the art. Some of available assays are based on the notion that development of dementia conditions is often associated with damage and/or loss of neuronal cells in a brain. In part, such damage and/or loss of neuronal cells may be caused due to some proteins being misfolded and/or aggregated in the cells. Thus, some of assays well known in the art are to measure cell death, protein aggregation and/or other associated indications to cell death and/or protein aggregation.

For example, as described in Blanchard and others (2004), one can have an assay that is configured to express amyloid precursor proteins (APP) in a cultured neuronal cell system. When overexpressed, the APP can be aggregated in the cell and can cause death of the cell. In a particular example, two or more fractions of the omentum tissue extract can be added to separate groups of the cultured neuronal cell expressing APP. Then the amount and/or time course of APP protein aggregation can be monitored and compared between cells treated with different fractions. Alternatively, cell death rate from separate groups of cells treated with different fractions can be measured and compared. When a certain group of cells treated with some fractions shows less APP aggregation or decrease in cell death, such some fractions can be further analyzed to identify a biological agent.

It is well known in the field that aggregation of a certain protein can be directly and/or indirectly monitored via various visualization techniques. For instance, APP protein aggregation can be directly monitored via microscopy such as electromicroscopy. Alternatively, APP can be labeled with a certain fluorescent dye or some other biomolecules so that monitoring such fluorescent dye or biomolecule can indirectly represent the degree of protein aggregation.

In addition, cell death can be monitored via various methods which are also available in the field. In one case, a certain dye such as trypan blue from Sigma-Aldrich Co. can be used to monitor cell death. Trypan blue can enter a cell if cell membrane has an opening and such opening may be present in a damaged or dead cell. In a petri-dish comprising cultured neuronal cells, cell death can be induced by expressing a certain protein such as APP in the cell. At least some of damaged or dead cells after expressing APP can be selectively counted by adding trypan blue dye to the petri-dish. When some fractions of the omentum tissue extract can cause decrease in cell death compared to other fractions, such some fractions can be further analyzed to identify a biological agent.

While a cell death and viability assay using trypan blue is described as an illustrative example herein, it should be apparent to a person skilled in the art that there are various modification and/or substitution in the illustrated procedures. Also it should be obvious that there are other methods available in the art for monitoring cell death and viability. Examples are BrdU (5-Bromo-2-deoxyuridine) assay, MTT (3-(4,5-Dimethylthiazol-2-yl)-2,5-diphenyltetrazolium bromide) assay, radioactive proliferation assay and others. Therefore any other assay configured to monitor cell death and/or viability in the field should be considered to be included in the scope of the disclosure.

In vitro assays generally refer to assays performed with a cultured cell and/or tissue, or in a cell-free system. The previously described assays of monitoring protein aggregation or cell death in a cultured cell system are some examples of in vitro assays. An alternative example of in vitro assays may include a biochemical assay that is substantially free of a living cell and/or tissue. For example, the protein aggregation can be tested within a test tube, which comprises a test protein and an appropriate solution. In some embodiments used in connection with the present disclosure, an amyloid precursor protein (APP) as described in Blanchard and other (2004) can be added into a test tube, wherein the test tube contains a certain buffer solution. When a plurality of such test tubes comprising APP proteins and a buffer solution are prepared, two or more fractions of the omentum tissue extract can be added to a separate test tube. When at least some fractions can decrease the aggregation of APP proteins compared to other fractions, such some fractions can be further analyzed to identify a biological agent. Monitoring the protein aggregation in a test tube can be done, for example, with microscopy. Alternatively, the protein aggregation in a test tube can be done through electrophoresis, irnmunohistochemical assay, circular dichroism (CD) spectroscopy and others.

In an alternative case, testing an activity of the fraction for an indication of its ability to treat dementia can be done in vivo. in vivo assays generally refer to assays that are performed in an animal partially or entirely. There are several disease models of dementia that are established with an animal such as a worm, a fruit fly, a mouse, and others. For example, a disease model using a fruit fly has been shown to recapitulate at least part of dementia conditions and reveal one or more symptom that is corresponding to symptoms present in human patients. As described in Jackson and others (1998), Gunawardena and others (2003) and Wolfgang and others (2005), a pathogenic form of a human protein associated with dementia can be expressed in a fruit fly compound eye through various genetic tools available in the art (e.g. Gal4-UAS system). In such examples, at least some neuronal cells in the fruit fly compound eye become damaged or lost resulting in the deformed eye. In this disease model, the potential activity of the fractions can be tested. In some illustrative example, a fruit fly having a deformed compound eye in the dementia disease model can be treated with the fraction of omentum tissue extract (e.g. a fraction of the omentum tissue extract can be fed to the fruit fly) and the effect on the eye morphology can be monitored as readout. When some fractions of the extract can rescue the abnormality of the fly eye development partially or completely, such some fractions can be further analyzed to identify a biological agent.

While a disease model using a fruit fly is described as an illustrative example of in vivo assays, it should be apparent to a person skilled in the art that a variety of modification and/or substitution can be made in the foregoing example. There are also other in vivo assays available in art to test an activity of the fractions for an indication of their ability to treat dementia, for example, measuring cognition function of mice from the disease model using mice. Therefore, the currently described assays and other available assays in the art that are configured to test the activity of the fraction for its ability to treat dementia should be included in the scope of the present disclosure.

In some embodiments, identification of a biological agent from the fraction of omentum tissue extract is provided. Such identification can be performed with one or more methods available in the art, such as a chromatographic technique, a mass spectrometry, an electrophoretic technique, and any combination thereof. In one example, the fraction that can decrease death of cultured neuronal cells induced by APP expression can be further analyzed with combinatorial techniques of an electrophoresis and a mass spectrometry. The fraction can be applied to 2-dimension (2D) gel electrophoresis to separate at least some proteins from each other on a native or denaturing gel. Separated proteins via 2D gel electrophoresis can be isolated from the gel via various methods such as dialysis. Once separated proteins are recovered from the gel, such proteins can be applied to a mass spectrometry with or without some treatment such as enzyme digestion. The mass spectrometry may be able to reveal the identity of at least some tested proteins or fragments thereof.

The activity of at least some identified proteins can be tested for an indication of their ability to treat dementia. Such activity test for the identified proteins can be done via a same or similar method that is described for testing an activity of the fraction elsewhere in this specification.

While the combinatorial method of an electrophoresis and a mass spectrometry is described as an illustrative example of identifying a biological agent from the fraction, it should be apparent to a person having ordinary skill in the art that the foregoing example can be modified and/or substituted in a variety way without abandoning the scope of the disclosure. Therefore, any modification and/or substitution in the illustrative example and also other methods well known in the art for identifying a biological agent from a fraction should be included in some embodiments.

In some other embodiments, administration of the omentum tissue or its extract to a mammal is provided. The omentum tissue or its extract can be obtained from a mammal or a cultured cell system as described in this specification. The omentum tissue or its extract can be administered to a mammal via a variety of means. Examples of such administration means include, but are not limited to, a means of oral administration, parenteral administration, inhalation, topical administration, rectal administration, vaginal administration, implantation, or any combinations thereof.

In one embodiment, a medical device that is configured to release a certain amount of the omentum tissue or the extract to a mammal is provided. The omentum tissue or the extract can be placed in the medical device before or after implantation. Alternatively, the medical device can obtain the omentum tissue or the extract from a mammal while it is present in the mammal. Such medical device can be implanted in an abdominal area and deliver the omentum tissue or the extract to a brain or other tissues. In addition, the medical device can be configured to release the omentum tissue or the extract to a vascular system such as blood vessels and/or lymph vessels. In one illustrative example, the omentum tissue or the extract thereof can be attached to the medical device which is coated with albumin and this medical device can be administered via subcutaneously, intramuscularly, and/or intravenously.

In some other embodiments, a composition comprising omentum tissue or its extract is provided. Omentum tissue in most embodiments generally refers to tissue comprising omentum. The omentum tissue or the extract of the omentum tissue can be obtained from a mammal or a cultured cell system. Some illustrative examples of how to obtain and/or prepare the omentum tissue or the extract are described elsewhere in the specification.

In some part of embodiments, the composition can further comprise at least one pharmaceutically acceptable carrier. The pharmaceutically acceptable carrier may include, but is not limited to, one or more of the followings: water, oil, albumin, a filler, a disintegrant, a lubricant, a glidant, and a binder.

In one aspect of embodiments, the composition targets symptoms and/or conditions associated with decline in cognition function. For example, the composition can be administered to a mammal to treat impaired memory and/or orientation, limitation of concentration, planning or judgment, personality changes, some disorders in perception, speech, and walking, some body dysfunction such as swallowing and/or excretion processes.

In addition, the composition can be administered to a mammal diagnosed with dementia and/or having symptoms associated with dementia. Dementia in this present disclosure may include, but is not limited to, Alzheimer's disease, Huntington's disease, Parkinson's disease, stroke, vascular dementia, Lewy body dementia, frontotemporal dementia, HIV-associated dementia, dementia pugilistica, corticobasal degeneration, Creutzfeldt-Jakob disease and others.

Another aspect of embodiments describes that the composition can be administered to a mammal via a variety of means. Examples of such administration means include, but are not limited to, a means of oral administration, parenteral administration, inhalation, topical administration, rectal administration, vaginal administration, implantation, or any combinations thereof. In one particular example, the composition can be administered parenterally, for example, via an injection to some area of a brain. Alternatively, the composition can be administered into vascular system such as blood vessels and/or lymph vessels. As such, the composition may be able to circulate the body and reach some area of a brain or other tissues where the composition may play a role to improve dementia conditions. In some other embodiments, the composition can be orally administered or implanted to a mammal.

In one illustrative example, the composition is configured to have a certain form of medicament such as a capsule, tablet, powder, gel, injection solution, patch, cream and others. In another illustrative example, a medical device that comprises the composition and is configured to release a certain amount of the composition to a mammal is provided. Such medical device can be implanted in an area of a brain. Alternatively, the medical device implanted in a mammal can be configured to release the composition to a vascular system such as blood vessels and/or lymph vessels so that the composition can be delivered to a brain or other tissues.

In another embodiment, a method of formulating the composition comprising the omentum tissue or the extract is described. One illustrative example of formulating method of the composition may comprise: providing an omentum tissue or an aqueous or organic extract of an omentum tissue, mixing the omentum tissue or the extract of an omentum tissue with at least one pharmaceutically acceptable carrier selected from the group consisting of water, oil, albumin, a filler, a disintegrant, a lubricant, a glidant, and a binder, and making a mixture of said omentum tissue or extract of an omentum tissue with at least one pharmaceutically acceptable carrier into a form suitable for oral administration, parenteral administration, inhalation, topical administration, rectal administration, vaginal administration, implantation, or any combinations thereof.

The omentum tissue or the extract thereof can be obtained from a mammal or a cultured cell system as described elsewhere in this specification. The omentum tissue or the extract thereof can be mixed with one or more pharmaceutically acceptable carrier. There are various pharmaceutically acceptable carriers in the art and selection of appropriate carriers can be done at least in part according to a certain administration means. Some non-limiting examples of a pharmaceutically acceptable carrier include water, oil, albumin, a filler, a disintegrant, a lubricant, a glidant, and a binder. Examples of administration means includes, but are not limited to, oral administration, parenteral administration, inhalation, topical administration, rectal administration, vaginal administration, implantation, or any combinations thereof.

In some embodiments, making the composition into a certain medicament form is described. In certain cases, the composition can be processed to be a tablet. In another case, the composition can be processed to be a gel, capsule, powder, cream, patch, injection material, and/or others.

In some other embodiments, a method of treating symptoms and/or conditions associated with dementia is provided. In certain cases, the method comprises administering the omentum tissue or the extract to a mammal. In some other cases, the method comprises administering the composition that comprises the omentum tissue or the extract to a mammal. Administration can be done in a variety of means such as oral administration, parenteral administration, inhalation, topical administration, rectal administration, vaginal administration, implantation, or any combinations thereof.

The symptoms and/or conditions that can be treated by the methods described herein include, but are not limited to, impaired memory and/or orientation, limitation of concentration, planning or judgment, personality changes, some disorders in perception, speech, and walking, some body dysfunction such as swallowing and/or excretion processes.

Dementia that can be treated by the methods described herein may include, but is not limited to, Alzheimer's disease, Huntington's disease, Parkinson's disease, stroke, vascular dementia, Lewy body dementia, frontotemporal dementia, HIV-associated dementia, dementia pugilistica, corticobasal degeneration, Creutzfeldt-Jakob disease and others.

In some other embodiments, a method of treating symptoms and/or conditions associated with dementia is provided, wherein the method comprises stimulating omentum in a mammal. In one example, a stimulant can be provided to a mammal in or near omentum. For instance, 1% lidocaine in the amount of two milliliters can be provided via injection to or near omentum. This injection may stimulate omentum and this can improve at least some symptoms associated with dementia. Such improvement may be at least in part because stimulation of omentum may induce production and/or secretion of a biological agent.

In an alternative example, omentum can be stimulated via a stimulator. In this particular case, a device configured to stimulate omentum mechanically, electrically and/or chemically can be implanted to or near omentum. This device can stimulate omentum by providing mechanical stimulus such as vibration, suction, incision of omentum and/or an electrical stimulus. Alternatively, the device can provide a stimulant such as 1% lidocaine to or near the omentum to induce chemical stimulation. In some embodiments, the stimulator can be implanted to a mammal in or near omentum. In one case, the entire stimulator is implanted in a mammal. In another case, some part of the stimulator can be implanted in a mammal and the rest of the stimulator may be present outside the mammal.

In some embodiments, the stimulator is implanted in or near omentum and further comprises one or more subunit, wherein at least one subunit is configured to obtain the omentum tissue and/or the extract from a mammal. This omentum tissue and/or the extract can be stored in another subunit that can function as a reservoir. Such omentum tissue and/or extract can be delivered to one or more tissue such as a brain. In one illustrative case, the delivery of the omentum tissue and/or its extract can be done through, for example, a catheter. In some embodiments, a catheter is connected to an implanted omentum stimulator and extends to a sub-dural spinal or sub-dural cranial tissue. This catheter can deliver the omentum tissue or its extract from the stimulator to the target tissue including a brain. In other embodiments, the catheter connected to the omentum stimulator extends to a subclavian or portal vein and/or lymph vessels so that the omentum tissue and/or its extract can enter the blood and/or lymph stream and be carried to the target tissue such as a brain. In certain embodiments, a pre-determined amount of the omentum tissue and/or its extract can be released from the stimulator and delivered to the target tissue at a regular time interval (e.g. once a day or twice per week) to certain tissues including a brain.

While several examples of how to stimulate omentum to improve dementia conditions are described above, those examples should be considered as only part of exemplary illustrations and should not be considered limiting the scope of the disclosure. It should be apparent to a person having ordinary skill in the art that a variety of modifications and/or substitutions can be made in any of foregoing described examples without abandoning the scope of the disclosure. Therefore, any modification and/or substitutions in the foregoing examples and any other methods available in the art that is configured to stimulate the omentum should be included to the scope of the disclosure.

What is claimed is:

1. A method of identifying a biological agent for treating dementia comprising:
obtaining an aqueous or organic extract of omentum tissue;
separating the extract into two or more fractions;
testing an activity of each of the fractions for an indication of its ability to treat dementia;
selecting at least one fraction that has the indication of ability to treat dementia; and
identifying one or more biological agents for treating dementia from said at least one fraction that has the indication of ability to treat dementia.

2. The method of claim 1, wherein said biological agent is a neurotrophic agent.

3. The method of claim 1, wherein said testing an activity of each of the fractions for an indication of its ability to treat dementia is done in vivo or in vitro.

4. A method of growing a mammalian omentum cell and/or tissue outside a mammal body comprising:
providing an omentum cell and/or tissue obtained from a mammal; and
growing the mammalian omentum cell and/or tissue in a cell culture system.

5. A composition comprising:
an omentum tissue or an aqueous or organic extract of an omentum tissue; and
at least one pharmaceutically acceptable carrier selected from the group consisting of water, oil, albumin, a filler, a disintegrant, a lubricant, a glidant, and a binder.

6. The composition of claim 5, wherein the omentum tissue or extract of an omentum tissue are obtained from a mammal or from a mammalian omentum cell culture system.

7. The composition of claim 5, wherein a target disease of said composition comprises dementia.

8. The composition of claim 5, wherein the composition is administered to a mammal via a means of oral administration, parenteral administration, inhalation, topical administration, rectal administration, vaginal administration, implantation, or any combinations thereof.

9. A method of formulating a composition of claim 5 comprising:
providing an omentum tissue or an aqueous or organic extract of an omentum tissue;
mixing the omentum tissue or the extract of an omentum tissue with at least one pharmaceutically acceptable carrier selected from the group consisting of water, oil, albumin, a filler, a disintegrant, a lubricant, a glidant, and a binder; and
making a mixture of said omentum tissue or extract of an omentum tissue with at least one pharmaceutically acceptable carrier into a form suitable for oral administration, parenteral administration, inhalation, topical administration, rectal administration, vaginal administration, implantation, or any combinations thereof.

10. A method of treating dementia comprising administering the composition of claim 5 to a mammal.

11. The method of claim 10, wherein the composition is administered to a mammal via a means of oral administration, parenteral administration, inhalation, topical administration, rectal administration, vaginal administration, implantation, or any combinations thereof.

12. A method of treating dementia comprising providing a stimulant in or near omentum to a mammal.

13. The method of claim 12, wherein the stimulant is 1% lidocaine.

14. A method of treating dementia comprising implanting an omentum stimulator to a mammal.

15. The method of claim 14, wherein the omentum stimulator is implanted in or near omentum.

16. The method of claim 14, wherein the stimulator is configured to deliver an omentum tissue and/or an aqueous or organic extract of an omentum tissue to a subcutaneous tissue, a subdural spinal tissue, a subdural cranial tissue or any combinations thereof.

17. A method of testing an effect of stimulating omentum on treating dementia comprises:
stimulating omentum in a first subject;
optionally stimulating tissue other than omentum in a second subject; and
measuring one or more symptom and/or condition related to dementia from the first and optional second subjects.

18. The method of claim 17, wherein said one or more symptom and/or condition related to dementia is measured via a behavioral approach, a neurological approach, a neuropsychological approach, a pathological approach, or any combinations thereof.

19. The method of claim 17, wherein stimulating omentum in the first subject is to apply a gastrostomy tube to or near omentum

20. The method of claim 17, wherein said stimulator in the second subject is an endoscopic tube and said endoscopic tube is provided through a mouth.
